SEQUENCE LISTING (5 sequences)

SEQ ID NO: 1 - protein, 28 aa, Artificial Sequence
  Synthetic Construct
CNLATCQLTCKSLGLLGKCAGSFCACGP

SEQ ID NO: 2 - protein, 27 aa, Artificial Sequence
  Synthetic Construct
XNLHFCQLRCKSLGLLGKCAGSXCACV

SEQ ID NO: 3 - protein, 32 aa, Artificial Sequence
  Synthetic Construct
MKQIEDKIEEILSKIYHIENEIARIKKLIGEV

SEQ ID NO: 4 - protein, 8 aa, Artificial Sequence
  Synthetic Construct
YLRDQQLL

SEQ ID NO: 5 - protein, 26 aa, Artificial Sequence
  Synthetic Construct
YIPEAPRDGQAYVRKDGEWVLLSTFL

(19) United States
(12) Patent Application Publication
(10) Pub. No.: US 2010/0221241 A1
(43) Pub. Date: Sep. 2, 2010

(54) CONSTRAINED HIV ENVELOPE-BASED IMMUNOGEN THAT SIMULTANEOUSLY PRESENTS RECEPTOR AND CORECEPTOR BINDING SITES

(75) Inventors: Anthony L. DeVico, Alexandria, VA (US); George Lewis, Baltimore, MD (US); Lai-Xi Wang, Ellicott City, MD (US)

Correspondence Address: MOORE & VAN ALLEN PLLC, P.O. BOX 13706, Research Triangle Park, NC, 27709, US

(73) Assignee: UNIVERSITY OF MARYLAND BIOTECHNOLOGY INSTITUTE, Baltimore, MD (US)

(21) Appl. No.: 11/994,870

(22) PCT Filed: Jul. 6, 2006
(86) PCT No.: PCT/US06/26136

§ 371 Date: Apr. 14, 2010

Related U.S. Application Data
(60) Provisional application No. 60/697,051, filed on Jul. 6, 2005.

(51) Int. Cl. A61K 39/395; C07K 14/00; A61K 39/00; A61P 31/18; A61P 37/04

(52) U.S. Cl. 424/130.1; 530/324; 424/185.1

(57) ABSTRACT

The present invention relates to a soluble binding complex comprising a soluble gp120 trimer, in which only two gp120 protomers have CD4 binding sites occupied by interconnecting CD4 mimetic moieties, thereby allowing for the exposure of CD4-induced epitopes on the mimetic-bound protomers and an unoccupied CD4 binding site on the third gp120 protomer.

### BACKGROUND OF THE INVENTION

1. Field of the Invention

The present invention generally relates to a HIV binding complex that exposes, simultaneously, the CD4 binding site on the HIV envelope gp 120 and the co-receptor binding site that is exposed only after gp120 and CD4 form a binding complex.

2. Background of the Related Art

A major goal for the development of HIV vaccines is to develop an HIV envelope (gp120)-based subunit immunogen that elicits broadly cross-reactive neutralizing antibodies that inhibit diverse natural strains of HIV-1 or HIV-2. To date, such an immunogen has not been developed and proven effective in humans.

It is known that the initial step of HIV entry is characterized by the interaction of HIV-1 envelope glycoprotein gp120 with host receptor CD4. The CD4 binding site on gp120 is centered on a conserved, hydrophobic pocket denoted the “Phe43 cavity.” It has been demonstrated that molecules targeting the conserved CD4-binding pocket, such as soluble CD4, CD4 mimetic proteins and HIV-neutralizing antibody b12, are potent inhibitors against HIV infection.

Although considerable effort has been expended on the design of effective therapeutics, currently no curative anti-retroviral drugs against AIDS exist. The new treatment regimens for HIV-1 or HIV-2 include a combination of anti-HIV compounds, which target reverse transcriptase (RI), such as azidothymidine (AZT), lamivudine (3TC), dideoxyinosine (ddl), tenofovir, nevirapine, efavirenz, or anti-HIV compounds which target HIV protease such as saquinavir, nelfinavir, indinavir, amprenavir, and lopinavir. Unfortunately, the development of viral resistance occurs in a significant number of treated patients using these compounds. This combined with the development of anti-retroviral drug induced toxicity continues to limit the overall impact of current available treatments. Thus, the toxicity and emergency of drug-resistant viruses associated with current drug regimen prompts development of new drugs that act with a different mode of action and with improved anti-HIV potency.

Effective envelope-based immunogens must raise broadly reactive antibodies that interfere in the HIV attachment and entry process. This process requires an interaction between the gp120 component of the trimeric envelope spike and cell surface CD4. Formation of the gp120-CD4 complex establishes viral attachment and induces the exposure of new gp120 epitopes including the entry coreceptor (normally a chemokine receptor) binding site. Thus, it is predicted that an effective subunit immunogen must: 1) present the structure of the native trimeric gp120 envelope found on virion surfaces; 2) present the CD4 binding site and/or 3) present epitopes induced by CD4 binding to gp120, including the coreceptor binding site. The ideal immunogen would manifest all three of these features.

However, no such molecule currently exists. Methods exist for producing trimeric envelopes, but these structures do not elicit broadly neutralizing antibodies. The CD4 binding sites on these structures are mainly unassembled (1) and the coreceptor binding domain is not presented. Recently, immunogens have been developed that combine gp120 with scorpion toxin-based CD4 mimetic miniproteins that induce the exposure of the coreceptor binding site. Although these immunogens raise broadly neutralizing antibodies, they do not present the CD4 binding site (the mimetic peptide occupies this site).

Thermodynamic studies (2) suggest that the gp120 components of the trimeric envelope spike “sample” a number of conformations in solution, only one of which is capable of interacting with CD4. Ultimately, CD4 snags the correct structure on one gp120 spike, which locks the trimer into a conformation that facilitates CD4 binding to the other two gp120s binding sites. If the binding process is completed, all of the gp120 spikes in the trimer are bound to CD4 and present CD4-induced epitopes. Heretofore, there has been no means to deliberately and predictably stop the process such that one CD4 binding site is assembled yet unoccupied.

Thus, it would be advantageous to develop an immunogen that exhibits a CD4 binding site while simultaneously exposing the co-receptor binding site that is exposed only after gp 120 and CD4 form a binding complex.

### SUMMARY OF THE INVENTION

In one aspect, the present invention relates to a soluble immunogen comprising a soluble gp120 trimer complexed to a bivalent molecule containing two CD4 mimetic moieties that positions the gp120 binding residues from about 3 to 7 nm, and more preferably, from about 4 to 6 nm apart.

In another aspect, the present invention relates to an isolated binding complex comprising a soluble gp120 trimer, in which only two gp120 protomers have CD4 binding sites occupied by a CD4 mimetic moiety, thereby allowing for the exposure of CD4-induced epitopes on the mimetic-bound protomers and an unoccupied CD4 binding site on the third gp120 protomer.

Preferably, the CD4 mimetic moiety comprises a molecule that binds to and has similar or enhanced affinity for the epitope on the gp120 protein that binds to CD4. For example, the CD4 mimetic may include, but is not limited to, CD4M9, CD4M33, BMS 378806, BMS. 488043 and similar small molecules that bind specifically to the CD4 binding sites located on the gp120 of the HIV virion.

Still another aspect of the present invention relates to a method of inhibiting entry of HIV in a cell, the method comprising:
introducing an effective amount of a binding complex comprising a soluble gp120 trimer, in which only two protomers have CD4 binding sites occupied by a CD4 mimetic moiety, thereby allowing for the exposure of CD4-induced epitopes on the mimetic-bound protomers and an unoccupied CD4 binding site on the third gp120 protomer.

Preferably, the mimetic moiety comprises monomeric units that are linked by a linker/spacer, wherein the linker/spacer is a sufficient length to position the monomeric units to bind to CD-4 binding sites on two adjacent gp120 protomers of a gp120 trimer. Preferably, the linker/spacer is a length for binding to reactive sites on the gp120 protomers and is in the range between about 3 to about 6 nm, and more preferably in the range between about 4 nm to 5.5 nm. The CD4 mimetic may include CD4M9, CD4M33, BMS 378806, BMS 488043 and similar small molecules that bind specifically to the CD4 binding sites located on the gp120 of the HIV virion.

Generally, the mimetic spacer/linker of the present invention may include any molecule that can bind and position the two CD4 mimetics at a sufficient distance to allow each of the mimetics to bind to a “Phe43 cavity” on two of the gp120 protomers. The mimetic spacer/linker may be cleavable or noncleavable, however preferably, the spacer/linker is noncleavable under physiological conditions and at a pH of from about 6.8 to about 7.5. The mimetic spacer/linker may include amino acid residues having a sufficient number to provide for a sequence that meets the distance requirements between the adjacent “Phe43 cavities” of adjacent gp120 protomers.

In yet another aspect, the present invention relates to a method of generating a binding complex that exposes a CD4 binding site on gp120 and at least one hidden epitope exposed with the binding of gp120 to CD4, the method comprising:
combining a soluble gp120 trimer with sub-saturating amounts of a CD4 mimetic moiety including CD4M9, CD4M33, synthesized compounds such as BMS378806, BMS488043 or any molecule that recognizes and targets for binding with the “Phe43 cavity” of gp120.

In the alternative, the immunogen could be produced by expressing stoichiometric ratios of single chain gp120-CD4 mimetic complexes and gp120 molecules from synthetic genes. In each case, two of the gp120-CD4 mimetic complexes and one gp120 molecule are linked together with a complexing moiety that facilitates trimer formation. The assembled immunogen would be composed of a trimer containing 1 or 2 gp120-CD4 mimetic complexes and 1 uncomplexed gp120.

In another aspect, the present invention relates to a method of generating broad neutralizing antibodies against HIV, the method comprising:
administering a binding complex comprising a soluble gp120 trimer, in which only two gp120 protomers have CD4 binding sites occupied by a CD4 mimetic molecule, thereby allowing for the exposure of CD4-induced epitopes on the mimetic-bound protomers and an unoccupied CD4 binding site on the third gp120 protomer.

Notably, this invention envisions that the binding complex may include a gp160 trimer that comprises three gp120/gp41 complexes, wherein the CD4 binding site of at least one of the gp120 protomers does not a CD4 mimetic molecule binding to the epitope and at one gp120 protomer having a CD4 mimetic molecule binding to the epitope. The three gp120 or gp120/gp140 protomers are linked together to form a soluble trimer. Clearly, the trimer is not part of a virion surface but instead is soluble and/or isolated.

In a still further aspect, the present invention relates to an HIV vaccine comprising a binding complex comprising a soluble gp120 timer, in which only two gp120 protomers have CD4 binding sites occupied by a CD4 mimetic molecule, thereby allowing for the exposure of CD4-induced epitopes on the mimetic-bound protomers and an unoccupied CD4 binding site on the third gp120 protomer.

In yet another aspect, the present invention relates to antibodies, including polyclonal and monoclonal, and production thereof, wherein the antibody is immunoreactive with a binding complex of the present invention.

In still a further aspect, the present invention contemplates a process for producing an antibody, which is immunoreactive with a binding complex of the present invention comprising the steps of:
(a) introducing the soluble binding complex into a live animal subject, wherein the soluble binding complex comprises a soluble gp120 trimer, in which only two gp120 protomers have CD4 binding sites occupied by a CD4 mimetic molecule, thereby allowing for the exposure of CD4-induced epitopes on the mimetic-bound protomers and an unoccupied CD4 binding site on the third gp120 protomer; and
(b) recovering antisera comprising antibodies specific for the binding complex.

Another aspect of the present invention relates to therapeutic HIV vaccine that exploits humoral immunity.

In another aspect, the present invention contemplates a diagnostic assay kit for detecting the presence in a biological sample of an immunoreactive antibody to the binding complex of the present invention, where the kit comprises a binding complex, which is capable of immunoreacting with antibodies in the biological sample, wherein the binding complex comprises a soluble gp120 trimer, in which only two gp120 protomers have CD4 binding sites occupied by a CD4 mimetic molecule, thereby allowing for the exposure of CD4-induced epitopes on the mimetic-bound protomers and an unoccupied CD4 binding site on the third gp120 protomer.

These and other aspects of the present invention, will be apparent from the detailed description of the invention provided hereinafter

### BRIEF DESCRIPTION OF THE FIGURES

FIG. 1 illustrates a binding complex immunogen of the present invention showing the binding orientation of the bivalent mimetic on the trimeric gp120 soluble protein.

### DETAILED DESCRIPTION OF THE INVENTION

In order to facilitate review of the various embodiments of the invention and provide an understanding of the various elements and constituents used in making and using the present invention, the following terms used in the invention description have the following meanings.

### DEFINITIONS

The term “spacer/linker” as used herein refers to a molecule that connects two monomeric units to form a bivalent molecule and still provides for binding of the bivalent molecule to the soluble gp120 trimer. Particular examples of spacer/linkers may include an amino acid spacer that is of sufficient length of residues to place the monomeric units in an appropriate spatial position to match the distance between the CD4 binding sites on the trimeric gp120. The amino acid spacer can essentially be any length, for example, as few as 5 or as many as 200 or more preferably from about 10 to 30 amino acid residues. Other examples of applicable spacer/linkers include a molecule that meets the distance requirements between the adjacent “Phe43 cavity” of gp120, including but not limited to the following bivalent molecules, such as bis-[β-(4-zaidosalicylamido)ethyl]disulfide; 1,4-bis-maleimidobutane; 1,4-bis-maleimidyl-2,3-dihydroxybutane; bis-maleimidohexane; bis-maleimidoethane; 1,8-bis-maleimidotriethyleneglycol; 1,11-bis-maleimidotetraethyleneglycol; bis[2-(Succinimidyloxycarbonyloxy)-ethyl]sulfone; bis[Sulfosuccinimidyl]suberate; disuccinimidyl glutarate; dithiobis(succinimidyl propionate); disuccinimidyl suberate; dithio-bis-maleimidoethane; 3, 3-dithiobis(sulfosuccinimidyl propionate); diethylene glycol bis(sulfosuccinimidylsuccinate); and dimethyl pimelimidate 2HCl.

The terms “peptide,” “polypeptide,” “miniprotein” and “protein” as used herein are used interchangeably to denote a sequence polymer of at least two amino acids covalently linked by an amide bond.

The term “therapeutic,” as used herein, means a treatment administered to a subject who exhibits signs of pathology for the purpose of diminishing or eliminating those signs.

The term “therapeutically effective amount,” as used herein means an amount of compound that is sufficient to provide a beneficial effect to the subject to which the compound is administered. A beneficial effect means rendering a virus incompetent for replication, inhibition of viral replication, inhibition of infection of a further host cell, or increasing CD4 T-cell count, for example.

The term “HIV” includes both HIV-1 and HIV-2 having envelope glycoprotein precursors, gp160 and gp140, respectively, which are cleaved to an external protein, gp120 and gp125, respectively and a transdomain protein, gp41 and gp36, respectively. For ease of understanding, we will typically refer to the HIV-1 precursor and subunits, but the skilled artisan can readily use the same methodology with HIV-2.

The term “gp120 trimer,” as used herein means a complex that mimics the native orientation of gp120s in the envelope spike, wherein the envelope spike may include three soluble HIV gp120 subunits; three gp120/gp41 heterodimers, wherein the gp120 and gp41 molecules can be from HIV or SIV virons; or three gp120 subunits fused together with a fusion linker, such as GCN4 (General Control Nonderepressible transcriptional activator of amino acid biosynthetic genes) a basic leucine zipper protein, into a trimerization motif to form the soluble trimer, and wherein the gp120 epitopes are not disrupted and recognized by neutralizing antibodies.

To create soluble forms of the HIV-1 envelope glycoproteins, the proteins may be truncated at various locations within the gp41 ectodomain. In addition, the natural cleavage site between the gp120 and gp41 glycoproteins may be altered to minimize proteolytic processing at this site. Although these two modifications result in soluble envelope glycoproteins, such proteins exhibit considerable heterogeneity, forming monomers, dimers, tetramers and other oligomers (6 and 7). Thus, to promote the formation of soluble trimers, a sequence from the GCN4 transcription factor that has modified to form trimeric coiled coils (8) may be appended to the carboxyl terminus of the soluble envelope glycoproteins (9 and 10).

One can use any coiled coil motif known to trimerize to stabilize the gp120 trimers. Coiled coil domains are comprised of heptad repeats with 2 characteristic hydrophobic amino acids found at the “a” and “d” residues of the helical wheel projection. Coiled coils may assemble into dimer, trimer, or tetramer bundles. The oligomeric form adopted by a particular coiled coil motif is largely a function of the amino acids found at the “a” and “d” positions, which form the inner core of the coiled coil bundle (8 and 11). gp41 contains coiled coil helices in its N terminus. In one preferred embodiment, the 32 amino acid trimeric motif of the leucine zipper protein GCN4 (MKQIEDKIEEILSKIYHIENEIARIKICLIGEV) (SEQ ID NO: 3) is fused C-terminal to the end ( . . . YLRDQQLL) (SEQ ID NO: 4) of the gp41 coiled coil, thereby extending the heptad repeat region and increasing the potential stability of trimer association. In another preferred embodiment, the 26 amino acid trimeric motif of the bacteriophage T4 fibritin trimeric (FT) sequence, ‘YIPEAPRDGQAYVRKDGEWVLLSTFL’ (SEQ ID NO: 5), is fused C-terminal to the end ( . . . YLRDQQLL) (SEQ ID NO:4) of the gp41 coiled coil, thereby extending the heptad repeat region and increasing the potential stability of trimer association.

Many trimeric motifs other than the GCN4 zipper domain and fibritin trimeric motif used here can be used in the gp41 portion to stabilize the gp120 trimer. Coiled coils, other than GCN4, represent one such example. Preferably, one uses 4-5 heptad repeats of a given coiled coil domain, approximately 30 amino acids. Such motifs may be chosen either from coiled coil domains found in known proteins or from peptides designed de novo. For example, the fusogenic protein hemagglutinin of influenza virus is one of the first identified and best characterized trimeric coiled coil proteins (12), with a coiled coil domain of 27 amino acids. Another well studied example is heat shock transcription factor, which contains a coiled coil motif of 71 amino acids (13). A large number of other proteins have been shown to contain trimeric coiled coil structures, including the 29 amino acid domain of vitamin-B12 receptor cubilin; paramyxovirus fusion protein, with a 38 amino acid domain; and vaccinia virus fusion protein, with a domain of 28 amino acids. These examples represent only a portion of the list of proteins which contain trimeric and trimeric coiled coils.

One also can introduce de novo designed trimeric and trimeric coiled coil motifs into the gp41 protein. Advances in protein design have begun to make it possible to specifically design a protein or peptide which adopts a predicted conformation. Among the successes in the field of de novo protein design have been peptides containing coiled coil motifs, including trimeric bundling peptides (8 and 12). Such peptides are typically around 30 amino acids and contain heptad repeats modeled on those found in known coiled coils, particularly with regard to the “a” and “d” amino acids, which direct assembly of the peptide helices into trimers (8).

The trimeric motif, such as a coiled coil can be inserted at the junction between the gp120/gp41 proteins, or just within the gp41 region. Typically, the remainder of the gp41 region will not be used. Other sites could also be used for the introduction of the coiled coils. In some embodiments, one can insert multiple coiled coils.

The term “CD4 mimetic,” as used herein, means any molecule that has binding affinity for CD4 binding site located on the gp120 of the HIV virion including, but not limited to, miniproteins CD4M9 and CD4M33 having the following respective sequences, or sequences having homology thereto and having similar functionality:

Amino acid sequences of the synthetic miniprotein CD4M9 and CD4M33, (SEQ ID NOs 1 and 2, respectively) as set forth below:

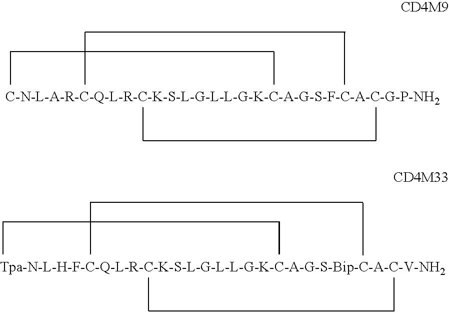

The sequences are shown by standard letter representations of amino acids, wherein CD4M33 contains two non-natural amino acids: Tpa, thiopropionic acid; Bip, biphenylalanine; and

small synthesized molecules such as BMS 378806 having the following structure:

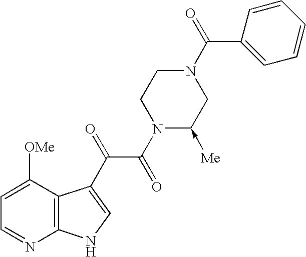

and other small molecules, such as scropion toxin, that can be joined in a linking fashion and occupy two CD4 binding sites on the gp120 trimer.

The present invention relates to a binding complex that comprises a soluble gp120 envelope protein and a bivalent molecule that occupies at least one CD4 binding sites on the native gp120 trimeric protein. Preferably, the bivalent molecule comprises two molecules that mimic the CD4 epitope and are covalently bonded or attached to a linker at a sufficient distance from each other to bind to two of the CD4 binding sites on the native gp120 trimeric protein.

The gp120/bivalent miniprotein binding complex of the present invention may be administered as a composition with various pharmaceutically acceptable carriers. Pharmaceutically acceptable carriers include those approved for use in animals and humans and include diluents, adjuvants, excipients or any vehicle with which a compound is administered. More specifically, pharmaceutically acceptable carriers include but are not limited to water, oils, saline, dextrose solutions, glycerol solutions, excipients such as starch, glucose, lactose, sucrose, gelatin, malt, rice, flour, chalk, silica gel, sodium stearate, glycerol monostearate, talc, sodium chloride, powdered non-fat milk, propylene glycol and ethanol. Pharmaceutical compositions may also include wetting or emulsifying agents, or pH buffering compounds.

In one embodiment, the present invention provides for compositions that include the binding complex of the present invention and optionally at least one additional antiviral agent, wherein the additional antiviral agent reduces replication of the HIV virus by a different mode of action. The compositions comprising a multivalent HIV inhibitor and optionally an additional antiviral agent, may be administered, separately, simultaneously, concurrently or consecutively.

The additional antiviral agent may include, but not limited to, nucleoside RT inhibitors, CCR5 inhibitors/antagonists, viral entry inhibitors and functional analogs thereof. Preferably, the antiviral agent comprises nucleoside RT inhibitors, such as Zidovudine (ZDV, AZT), Lamivudine (3TC), Stavudine (d4T), Didanosine (ddl), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC);

CCR5 inhibitors/antagonists, such as SCH-C, SCH-D, PRO 140, TAK 779, TAK-220, RANTES analogs, AK602, UK-427, 857, monoclonal antibodies;

viral entry inhibitors, such as Fuzeon (T-20), NB-2, NB-64, T-649, T-1249, SCH-C, SCH-D, PRO 140, TAK 779, TAK-220, RANTES analogs, AK602, UK-427, 857; and functional analogs thereof.

The compositions and methods of the present invention can be used to treat or reduce effects of HIV viral infection in a subject potentially exposed to the infection. The binding complex of the present invention may be administered for the treatment of HIV either as a single therapeutic agent or in combination with other antiretroviral drugs that attack the virus at different points of replication.

A composition of the present invention is typically administered parenterally in dosage unit formulations containing standard, well-known nontoxic physiologically acceptable carriers, adjuvants, and vehicles as desired. The term parenteral as used herein includes intravenous, intramuscular, intraarterial injection, or infusion techniques.

Injectable preparations, such as sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables. Preferred carriers include neutral saline solutions buffered with phosphate, lactate, Tris, and the like.

The compositions of the invention are administered in substantially non-toxic dosage concentrations sufficient to ensure the release of a sufficient dosage unit of the present complexes into the patient to provide the desired antibody production with subsequent inhibition of the HIV virus. The actual dosage administered will be determined by physical and physiological factors such as age, body weight, severity of condition, and/or clinical history of the patient. The active ingredients are ideally administered to achieve in vivo plasma concentrations of about 0.01 uM to about 100 uM, more preferably about 0.1 to 10 uM. It will be understood, however, that dosage levels that deviate from the ranges provided may also be suitable in the treatment of a given viral infection.

Therapeutic efficacy of the binding complex can be determined by standard pharmaceutical procedures in cell cultures or experimental animals, e.g., for determining The LD₅₀ (The Dose Lethal To 50% Of The Population) and The ED₅₀ (the dose therapeutically effective in 50% of the population). The dose ratio between toxic and therapeutic effects is the therapeutic index and it can be expressed as the ratio LD₅₀/ED₅₀. Compounds, which exhibit large therapeutic indexes, are preferred. The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the form of dosage employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutic compositions according to the present invention may be employed in combination with other-therapeutic agents for the treatment of viral infections or conditions. Examples of such further therapeutic agents include agents that are effective for the treatment of viral infections or associated conditions such as immunomodulatory agents such as thymosin, ribonucleotide reductase inhibitors such as 2-acetylpyridine 5-[(2-chloroanilino) thiocarbonyl)thiocarbonohydrazone, interferons such as alpha-interferon, 1-beta-D-arabinofuranosyl-5-(1-propynyl)uracil, 3′-azido-3′-deoxythymidine, ribavirin and phosphonoformic acid.

In an alternate embodiment, the present invention provides expression vectors comprising polynucleotides that encode the binding complex including the gp 120 trimeric protein and CD4 mimetic. The CD4 mimetic may be expressed as the bivalent molecule that includes an amino acid linker between CD4 mimetics. Preferably, expression vectors of the invention comprise polynucleotides operatively linked to an enhancer-promoter. More preferably still, expression vectors of the invention comprise a polynucleotide operatively linked to a prokaryotic or eukaryotic promoter.

A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

Expression of the gp120 may be effected in a gp120 expression plasmid such as YUgp140 wherein the gp41 may or may not be deleted. The expression vector may be included in different cell lines for expression including S2 cell lines. Means of transforming or transfecting cells with exogenous polynucleotide such as DNA molecules are well known in the art and include techniques such as calcium-phosphate- or DEAE-dextran-mediated transfection, protoplast fusion, electroporation, liposome mediated transfection, direct microinjection and adenovirus infection.

The most widely used method of transfection is mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

A transfected cell can be prokaryotic or eukaryotic. Preferably, the host cells of the invention are eukaryotic host cells.

In addition to prokaryotes, eukaryotic microbes, such as yeast can also be used. Saccharomyces cerevisiae or common baker's yeast is the most commonly used among eukaryotic microorganisms, although a number of other strains are commonly available. For expression in Saccharomyces, the plasmid YRp7, for example, is commonly used. This plasmid already contains the trp1 gene, which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan. Suitable promoter sequences in yeast vectors include the promoters for 3-phosphoglycerate kinase or other glycolytic enzymes such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also introduced into the expression vector downstream from the sequences to be expressed to provide polyadenylation of the mRNA and termination. Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin or replication and termination sequences is suitable.

In addition to microorganisms, cultures of cells derived from multicellular organisms can also be used as hosts. In principle, any such cell culture is workable, whether from vertebrate or invertebrate culture. However, interest has been greatest in vertebrate cells, and propagation of vertebrate cells in culture (tissue culture) has become a routine procedure in recent years. Examples of such useful host cell lines are AtT-20, VERO and HeLa cells, Chinese hamster ovary (CHO) cell lines, and W138, BHK, COSM6, COS-1, COS-7, 293 and MDCK cell lines. Expression vectors for such cells ordinarily include (if necessary) an origin of replication, a promoter located upstream of the gene to be expressed, along with any necessary ribosome binding sites, RNA splice sites, polyadenylation site, and transcriptional terminator sequences.

For use in mammalian cells, the control functions on the expression vectors are often derived from viral material. For example, commonly used promoters are derived from polyoma, Adenovirus 2, Cytomegalovirus and most frequently Simian Virus 40 (SV40). The early and late promoters of SV40 virus are particularly useful because both are obtained easily from the virus as a fragment, which also contains the SV40 viral origin of replication. Smaller or larger SV40 fragments can also be used, provided there is included the approximately 250 by sequence extending from the HindIII site toward the BglI site located in the viral origin of replication. Further, it is also possible; and often desirable, to utilize promoter or control sequences normally associated with the desired gene sequence, provided such control sequences are compatible with the host cell systems.

Following transfection, the cell is maintained under culture conditions for a period of time sufficient for expression of the component peptides of the present invention. Culture conditions are well known in the art and include ionic composition and concentration, temperature, pH and the like. Typically, transfected cells are maintained under culture conditions in a culture medium. Suitable medium for various cell types are well known in the art. In a preferred embodiment, temperature is from about 20° C. to about 50° C. pH is preferably from about a value of 6.0 to a value of about 8.0, more preferably from about a value of about 6.8 to a value of about 7.8 and, most preferably about 7.4. Other biological conditions needed for transfection and expression of an encoded protein are well known in the art. Transfected cells are maintained for a period of time sufficient for expression of the peptide. A suitable time depends inter alia upon the cell type used and is readily determinable by a skilled artisan. Typically, maintenance time is from about 2 to about 14 days.

A recombinant the gp120 trimer protein/bivalent peptide is recovered or collected either from the transfected cells or the medium in which those cells are cultured. Recovery comprises isolating and purifying the recombinant polypeptide. Isolation and purification techniques for polypeptides are well known in the art and include such procedures as precipitation, filtration, chromatography, electrophoresis and the like.

In still another embodiment, the present invention provides antibodies immunoreactive with the binding complex of the present invention. Preferably, the antibodies of the invention are monoclonal antibodies. Means for preparing and characterizing antibodies are well known in the art.

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Methods for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions. Adjuvants that may be used include IL-1, IL-2, IL-4, IL-7, IL-12, .gamma.-interferon, GMCSP, BCG, aluminum hydroxide, MDP compounds, such as thur-MDP and nor-MDP, CGP (MTP-PE), lipid A, and monophosphoryl lipid A (MPL). MI, which contains three components extracted from bacteria, MPL, trehalose dimycolate (TDM) and cell wall skeleton (CWS) in a 2% squalene/Tween 80 emulsion. Exemplary, often preferred adjuvants include complete Freund's adjuvant (a non-specific stimulator of the immune response containing killed Mycobacterium tuberculosis), incomplete Freund's adjuvants and aluminum adjuvants.

In addition to adjuvants, it may be desirable to coadminister biologic response modifiers (BRM), which have been shown to upregulate T cell immunity or down-regulate suppressor cell activity. Such BRMs include, but are not limited to, Cimetidine (CIM; 1200 mg/d) (Smith/Kline, PA); or low-dose Cyclophosphamide (CYP; 300 mg/m.sup.2) (Johnson/Mead, N.J.) and cytokines such as .gamma.-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intranasal, intravenous and intraperitoneal). The production of polyclonal antibodies may be monitored by sampling blood of the immunized animal at various points following immunization. A second, booster injection, may also be given. The process of boosting and titering is repeated until a suitable titer is achieved. When a desired level of immunogenicity is obtained, the immunized animal can be bled and the serum isolated and stored, and/or the animal can be used to generate MAbs.

MAbs may be readily prepared through use of well-known techniques. Typically, this technique involves immunizing a suitable animal with a selected immunogen composition, e.g., a purified or partially purified soluble binding complex of the present invention. The immunizing composition is administered in a manner effective to stimulate antibody producing cells. The methods for generating monoclonal antibodies (MAbs) generally begin along the same lines as those for preparing polyclonal antibodies. Rodents such as mice and rats are preferred animals, however, the use of rabbit, sheep frog cells is also possible. The use of rats may provide certain advantages but mice are preferred, with the BALB/c mouse being most preferred as this is most routinely used and generally gives a higher percentage of stable fusions. The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blobd sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art. For example, where the immunized animal is a mouse, one may use P3-X63/Ag8, X63-Ag8.653, NS1/1.Ag 41, Sp210-Ag14, FO, NSO/U, MPC-11, MPC11-X45-GTG 1.7 and S194/5XX0 Bul; for rats, one may use R210.RCY3, Y3-Ag 1.2.3, 1R983F and 4B210; and U-266, GM1500-GRG2, LICR-LON-HMy2 and UC729-6 are all useful in connection with human cell fusions. One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods include using Sendai virus and polyethylene glycol (PEG). Further, the use of electrically-induced fusion methods is also appropriate.

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like. The selected hybridomas would then be serially diluted and cloned into individual antibody-producing cell lines, which clones can then be propagated indefinitely to provide MAbs. The cell lines may be exploited for MAb production in two basic ways.

A sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. MAbs produced by either means may be further purified, if desired, using filtration, centrifugation and various chromatographic methods such is HPLC or affinity chromatography.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature and the contents of which are include herein by reference for all purposes. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### Example 1

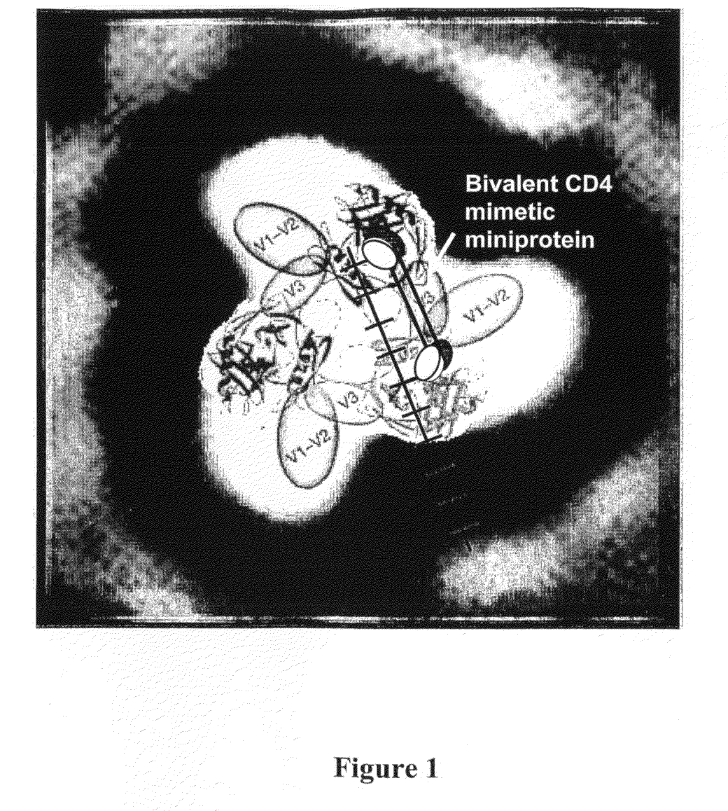

Recently, the X-ray crystal structure of free gp120 was solved (1) and along with X-ray tomography images of envelope spikes (3), used to build a computational model of the envelope trimer (1). This model indicates that any two CD4 binding sites in the envelope trimer are separated by roughly 4-5 nm. The model is overlayed with a ruler as shown in FIG. 1. The line between the binding sites is a “ruler” divided into 1 nm units, which was used to estimate distances between binding sites. Recently, the present inventors showed that bivalent polypeptides containing two CD4M9 moieties separated by polyethylene glycol linkers that placed the binding residues 5.2 nm (bi-CD4M9-MS) or 6.4 nm (bi-CD4M9-LS) apart demonstrated more potent HIV inhibition than monomeric CD4M9 (4 and 5) when used with surface HIV virion. Recent studies using Surface Plasmon Resonance to assess binding to trimers showed that the forward rate constant for binding of bi-CD4M9-MS was significantly different from monomeric CD4M9.

Two CD4M9 monomeric molecules can be linked with a bivalent compound such a bis-maleimide by dissolving the CD4M9 molecules in degassed phosphate buffer (50 mM, pH 6.6) containing 50% acetonitrile. Then a solution of bis-maleimide compounds of differing lengths may be added. To match the distance between any two of the CD4-binding cavities, bivalent molecules are designed by tethering two CD4M9 moieties through a linker of varied length. For constructing such a covalently-linked bivalent molecule, an extra cysteine residue may be introduced at the C-terminus of CD4M9 as a tag for late-stage chemoselective ligation, because the model of the interaction between CD4M9 and gp120 suggested that the C-terminus of CD4M9 did not have any contact with residues of gp120 and was pointed away from the binding sites (14).

The length and flexibility of the linker are critical to place the two CD4M9 moieties in an appropriate spatial position to match the two binding sites in the trimeric gp120, and as such, the optimal length may be determined by probing with different lengths of the linker. Acceptable bis-maleimide compounds include 1, 8-bis-(6-maleimidocaproylamido)-triethyleneglycol or 1,8-bis-[6-(4-N-maleimidomethyl-cyclohexane-1-carboxyl)amido]caproxyl-triethyleneglycol. The products can be purified by HPLC and characterized by ESI-MS. The maximal distance of the two Phe-ligands in the synthetic bivalent ligands preferably falls into the estimate range of 30-60 Å between any two of the three “Phe43 cavities” in the modeled trimeric gp120 complex.

The soluble gp120 trimer and the CD4M9 linked bivalent molecule are mixed for a sufficient time and under acceptable conditions for the binding of the CD4 mimetics to the CD4 binding site on two on the gp120 protomers in the trimer complex.

To confirm that the binding complex of the present invention exposes multiple epitope both exposed (gp120) and hidden (exposed only after binding of CH4 mimetic to Gp120 binding site) the complex may be exposed to known and commercially available antibodies that have binding affinity for such epitopes.

Further, the complexes may be evaluated for antibody production and anti-HIV activity. The methods generally involve administering to an animal a pharmaceutical composition comprising an immunologically effective amount of the binding complexes of the present invention that is capable of generating an immune response in the recipient animal, that includes both the generation of an antibody response (B cell response), and/or the stimulation of a cytotoxic immune response (T cell response). The generation of such an immune response will have utility in both the production of useful bioreagents, e.g., CTLs and, more particularly, reactive antibodies, for use in prophylactic or therapeutic embodiments. Preferred animals include mammals, and particularly humans. Other preferred animals include murines, bovines, equines, porcines, canines, felines and non-human primates.

### REFERENCES

The contents of the following references are hereby incorporated by reference herein for all purposes.
1. Chen B., Vogan, E. M., Gong H., Skehel, J. J., Wiley, D. C. and Harrison, S. J. 2005 Structure of the unliganded simian immunodeficiency virus pp 120 core. Nature 433: 834-841.
2. Kwong, P. D. et al 2002 HIV-1 evades antibody-mediated neutralization through conformational masking of receptor-binding sites. Nature 420: 678-682.
3. Zhu, P., Chertova, E., Bess, J., Lifson, J. D., Arthur, L. O., Liu, J, Taylor, K. A., and Roux, K. H., 2003 Electron tomography analysis of envelope glycoprotein trimers on HIV and simian immunodeficiency virus virions. PNAS 100:15812-15817.
4. Li, H., Song, H., Heredia, A., Le N., Redfield, R., Lewis, G. K. and Wang, L. X., 2004 Synthetic bivalent CD4-mimetic miniproteins show enhanced anti-HIV activity over the monovalent miniprotein. Bioconjugate Chemistry 15: 783-789.
5. U.S. patent application Ser. No. 11/054,398, filed on Feb. 9, 2005, titled ENHANCING ANTI-HIV EFFICIENCY THROUGH MULTIVALENT INHIBITORS TARGETING OLIGOMERIC GP120.
6. Earl P. L., Broder C. C., Long D., et al. 1994, Native oligomeric human-immunodeficiency-virus type-1 envelope glycoprotein elicits diverse monoclonal-antibody reactivities, J. Virol. 68: 3015-3026.
7. Earl P. L., Doms R. W., Moss B., 1990, Oligomeric structure of the human immunodeficiency virus type-1 envelope glycoprotein, Proc. Natl. Acad. Sci. USA 87: 648-652.
8. Harbury P. B., Zhang T., Kim P. S., et al., 1993, A switch between 2-stranded, 3-stranded and 4-stranded coiled coils in gcn4 leucine-zipper mutants, Science 262: 1401-7.
9. Yang X., Florin L., Farzan M., Kolchinsky P., Kwong P., Sodroski J., and Wyatt R., 1999, Modifications That Stabilize Human Immunodeficiency Virus Envelope Glycoprotein Trimers in Solution, J. Virol. 74: 4746-4754.
Yang X., Farzan M., Wyatt R., and Sodroski J., 2000, Characterization of Stable, Soluble Trimers Containing Complete Ectodomains of Human Immunodeficiency Virus Type 1 Envelope Glycoproteins, J. Virol. 74: 5716-5725, 2000).
11. Wagschal K., Tripet B., Hodges R. S., 1999, De novo design of a model peptide sequence to examine the effects of single amino acid substitutions in the hydrophobic core on both stability and oligomerization state of coiled-coils, J. Mol. Biol. 285: 785-803.
12. Wilson I. A., Skehel J. J., Wiley D. C., 1981, Structure of the hemagglutinin membrane glycoprotein of influenza-virus at 3-a resolution, Nature 289: 366-73.
13. Nomizu M., Utani A., Shiraishi N., et al., 1992, Synthesis and conformation of the trimeric coiled-coil segment of laminin, Int. J. Pept. Protein Res. 40: 72-9.
Vita, C., Drakopoulou, E., Vizzavona, J., Rochette, S., Martin, L., Menez, A., Roumestand, C., Yang, Y. S., Ylisastigui, L., Benjouad, A., and Gluckman, J. C. (1999) Rational engineering of a miniprotein that reproduces the core of the CD4 site interacting with HIV-1 envelope glycoprotein. Proc. Natl. Acad. Sci. USA 96, 13091-13096.

That which is claimed is:

1. A soluble binding complex comprising a soluble gp120 trimer comprising three gp120 protomers complexed to a bivalent molecule, wherein the bivalent molecule comprises two CD4 mimetic moieties that bind to CD4 binding sites on adjacent gp120 protomers of the gp120 trimer.

2. The soluble binding complex according to claim 1, wherein the bivalent molecule further comprises a linker/spacer for positioning the two CD4 mimetic moieties from about 3 nm to about 6 nm apart.

3. The soluble binding complex according to claim 1, wherein an unoccupied CD4 binding site remains on the third gp120 protomer.

4. The binding complex according to claim 2, wherein the CD4 mimetic moieties comprise CD4M9 molecules linked by a linker/spacer molecule of sufficient length to provide for binding to the CD-4 binding sites on the gp120 trimer.

5. The binding complex according to claim 4, wherein the linker/spacer is sufficient length to place the two CD4M9 from about 4 nm to about 5 nm apart when positioned on two of the protomers of the gp120 trimer.

6. The binding complex according to claim 2, wherein the linker/spacer is an amino acid sequence.

7. The binding complex according to claim 2, wherein the linker/spacer is a bis-maleimide compound.

8. The binding complex according to claim 7, wherein the amino acid sequence is of sufficient length of residues to place the monomeric units in an appropriate spatial position to match the distance between the CD4 binding sites on the trimeric gp120.

9. The binding complex according to claim 1, further comprising a coiled coil motif known to trimerize for stabilizing the gp120 trimer.

10. A method of generating a binding complex that exposes a CD4 binding site on gp120 and at least one hidden epitope expose with the binding of gp120 to CD4, the method comprising:
combining a soluble gp120 trimeric complex comprising three gp120 protomers with sub-saturating amounts of a bivalent molecule, wherein the bivalent molecule comprises two CD4 mimetic moieties that bind to CD4 binding sites on adjacent gp120 protomers of the gp120 trimer.

11. The method according to claim 10, wherein the CD4 mimetic molecule is CD4M9, CD4M33, BMS378806, or BMS488043.

12. The method according to claim 10, wherein the bivalent molecule further comprises a linker/spacer for positioning the two CD4 mimetic moieties from about 3 nm to about 6 nm apart.

13. The method according to claim 10, wherein an unoccupied CD4 binding site remains on the third gp120 protomer.

14. The method according to claim 12, wherein the CD4 mimetic moieties comprise CD4M9 molecules linked by a linker/spacer molecule of sufficient length to provide for binding to the CD-4 binding sites on the gp120 trimer.

15. The method according to claim 14, wherein the linker/spacer is sufficient length to place the two CD4M9 from about 4 nm to about 5 nm apart when positioned on two of the protomers of the gp120 trimer.

16. The method according to claim 12, wherein the linker/spacer is an amino acid sequence.

17. The method according to claim 12, wherein the linker/spacer is a bis-maleimide compound.

18. The method according to claim 17, wherein the amino acid sequence is of sufficient length of residues to place the monomeric units in an appropriate spatial position to match the distance between the CD4 binding sites on the trimeric gp120.

19. The method according to claim 10, further comprising a coiled coil motif known to trimerize for stabilizing the gp120 trimer.

20. A method of generating broad neutralizing antibodies against HIV, the method comprising:
(a) administering a binding complex to a mammal, the binding complex comprising a soluble gp120 trimer comprising three gp120 protomers complexed to a bivalent molecule, wherein the bivalent molecule comprises two CD4 mimetic moieties that bind to CD4 binding sites on adjacent gp120 protomers of the gp120 trimer; and
(b) recovering antisera comprising antibodies specific for the binding complex.

21. The method according to claim 20, wherein the bivalent molecule further comprises a linker/spacer for positioning the two CD4 mimetic moieties from about 3 nm to about 6 nm apart.

22. The method according to claim 20, wherein an unoccupied CD4 binding site remains on the third gp120 protomer.

23. The method according to claim 21, wherein the CD4 mimetic moieties comprise CD4M9 molecules linked by a linker/spacer molecule of sufficient length to provide for binding to the CD-4 binding sites on the gp120 trimer.

24. The method according to claim 23, wherein the linker/spacer is sufficient length to place the two CD4M9 from about 4 nm to about 5 nm apart when positioned on two of the protomers of the gp120 trimer.

25. The method according to claim 23, wherein the linker/spacer is an amino acid sequence.

26. The method according to claim 23, wherein the linker/spacer is a bis-maleimide compound.

27. The method according to claim 25, wherein the amino acid sequence is of sufficient length of residues to place the monomeric units in an appropriate spatial position to match the distance between the CD4 binding sites on the trimeric gp120.

28. The method according to claim 20, further comprising a coiled coil motif known to trimerize for stabilizing the gp120 trimer.

29. A method of generating an immune response to reduce the effects of HIV, the method comprising administering to a mammal a therapeutic HIV vaccine comprising a soluble gp120 trimer having three gp120 protomers, in which only two protomers have CD4 binding sites occupied by a CD4 mimetic miniprotein, thereby allowing for the exposure of CD4-induced epitopes on the mimetic-bound protomers and an unoccupied CD4 binding site on the third gp120 protomer.
